(19) United States
(12) Patent Application Publication
(10) Pub. No.: US 2008/0188775 A1
(43) Pub. Date: Aug. 7, 2008

(54) Force Evaluating Device and a Force Evaluating Method for Determining Balance Characteristics

(76) Inventor: Peter Schneider, Wurzburg (DE)

Correspondence Address: FAY KAPLUN & MARCIN, LLP, 150 BROADWAY, SUITE 702, NEW YORK, NY, 10038, US

(21) Appl. No.: 11/630,942

(22) PCT Filed: Mar. 15, 2005
(86) PCT No.: PCT/DE2005/000467

§ 371 Date: Nov. 9, 2007

(30) Foreign Application Priority Data
Jul. 3, 2004 (DE) 102004032398.4

(51) Int. Cl. A61B 5/103

(52) U.S. Cl. 600/592

(57) ABSTRACT

The present invention relates to a force evaluating device with which forces can be determined and evaluated in a time-resolved manner. It has at least three force inducers which are arranged in a plane and which are coupled to a support plate a person can walk on. The compressive force acting on the force inducers via the support plate can be detected in a time-resolved manner by means of the force inducers. The power spectrum of the forces is calculated from the detected measured values by means of an evaluation unit. Furthermore, the determined force/time relationship by time can be differentiated from which further characteristics of the acting forces can be determined. The areas of application of the invention are mainly the field of health care and the field of rehabilitation measures and the field of the objectification of the influence of alcohol and drugs on human reaction capabilities.

### FIELD OF INVENTION

The present invention relates to a device and to a method for determining balance characteristics. The device in accordance with the invention in particular serves for the quantitative detection of functions and of disorders in the motor and sensory balance system in humans.

### BACKGROUND INFORMATION

The balance of a standing human is ensured by the harmonious interaction of the motor system and sensory system of the human musculoskeletal system and by special functions of the central nervous system, the spinal motor system and the supraspinal motor system. Standing balance is currently investigated by different tests which only permit a subjective assessment by the observer, e.g. using a stopwatch.

Problems in the spinal motor system and in the supraspinal motor system are, on the one hand, caused by diseases or injuries; on the other hand, they are subject to ageing processes. All the components which ensure a secure standing position or unerring voluntary and involuntary movements can be affected thereby. In the final result, the fine regulation of the muscle groups of the lower extremities, in particular of the flexor of the foot and of the extensor of the foot, lead to the maintenance of balance while standing.

The diagnosis of an osteopenia and of an osteoporosis is today carried out using bone density measurements and does not take account of either the causes or the pathogenesis or the bone strength. The occurrence of osteoporosis-related fractures in women after the menopause is thus mainly considered due to reduced bone strength. The bone density is therefore determined as a preventive measure to determine the risk of suffering a fracture. In fact, however, the probability of a fracture is more than 80% dependent on other factors. The following factors can inter alia be counted among them: reduction in the strength or the muscular performance of the lower extremities, balance disorders, gait disorders, taking of medication, alcohol and other drugs, cognitive reduction with respect to the environment and reduction in visual acuity. These factors in part represent health impairments. They are currently determined only in qualitative form (anamnesis). It is generally also not taken into account that due to our civilization society a reduced strain on the musculoskeletal system results in a physiological adaptation process which contributes to the probability of fracture. Numerous studies document that current physical activity is accompanied by a clear reduction in the risk of fracture in men and women. What is also decisive here is the level of physical activity (see, for example, Karlsson, J. in: Musculoskelet. Neuron. Inter., 4/2004, pages 12 to 21).

The risk of a fall of a person is closely linked to the sensomotoric regulation of the center of gravity. Numerous endogenic and exogenic factors have an influence on the performance of the sensomotoric system. The spinal and supraspinal motor systems can be influenced by pathological processes or changes due to ageing at different positions. They can thus have performance deficits which are accompanied by an increased risk of falling. The degree and the speed at which muscular performance can be activated to correct an excursion of the center of gravity in the direction of an unstable state and to avoid a fall are expressed in this.

The epidemiological data permit conclusions to be drawn from the frequency of fractures on the underlying mechanical properties of the bones and other factors which can be associated therewith. A relationship to the probability of fractures can be derived by means of the bone density as a surrogate parameter or replacement parameter for the bone strength. It is shown in statistical model calculations on the basis of large epidemiological studies that the bone density can only explain approximately 15% of the probability of a fracture. If a fracture is not primarily the result of an unavoidable and unforeseen effect of force, a disturbance from the outside can be countered by means of the protective mechanism of fall avoidance or of the mechanism of a suitable evasive reaction. A measure for the capability of this should generally be able to be derived from a simple system of balance analysis.

However, no apparatus exists with which the fall probability and thus the risk of fracture can be determined with respect to the aforesaid factors (reduction in strength or muscular performance of the lower extremities, balance disorders, gait disorders, influence of medication, influence of alcohol and/or other drugs, cognitive reduction with respect to the environment and/or reduction in visual acuity).

### SUMMARY OF INVENTION

The present invention relates to an apparatus and a method with which the forces exerted and/or the movements made by a person for the maintenance of balance can be quantitatively detected. The present invention also relates to an apparatus and a method with which physical characteristics can be derived from the quantitatively detected forces which characterize the movement behavior and/or strength behavior of the person.

The present invention is based on the quantitative detection of the control capability of the motoric and sensory system. The known tandem stand test is used for this purpose. In the tandem stand test (also called the tandem test or tandem stand in the following), the capability is tested of being able to maintain a stance for approximately 10 s with the feet arranged in front of one another (heel of the one foot arranged directly in front of the forward section of the other foot or both feet arranged on a straight line) without taking a step to the side.

The force evaluating device in accordance with the invention has at least three force inducers or force sensors arranged in a plane (force inducer plane). A rigid, walkable support plate is coupled to these force inducers. If a compressive force (for example the weight of a person) is exerted on this support plate on the side remote from the force inducers during a time interval (measurement interval), the force inducers, which are decoupled from one another, pick up the forces transmitted to the force inducers via the support plate and resulting on the basis of the compressive force in a time-resolved manner in each case. An evaluation unit is connected to the force inducers at the signal output side. It has a power spectrum calculation unit with which the power spectrum of a total signal can be calculated which is formed from a sum of the output signals of the force inducers. In this connection, the total signal can be formed from the sum of the output signals of all force inducers; it is, however, also possible only to form the sum via some of the force inducers or of the associated output signals. The output signal of an individual force inducer can also be used as the total signal. Alternatively or additionally, the force evaluating device in accordance with the invention has a differentiation unit with which the time derivation of the total signal just described can be calculated and with which further characteristics can be derived from the calculated time derivation.

In a preferred embodiment, exactly three force inducers are used which are arranged in the plane of the force inducers in the form of a triangle. It is particularly preferably an arrangement in the form of an equilateral triangle.

In a further advantageous embodiment, the force evaluating device has a base plate on the side remote from the support plate and adjacent to the force inducers. The unit of support plate and base plate, with which the force inducers are then arranged, can thus be installed on a planar base surface in a stable, robust and simple manner.

In a further advantageous embodiment variant, the evaluation unit has operational amplifiers which are connected to the force inducers at the signal output side and with which the output signal of the force inducers can be amplified. The evaluation unit then has an analog-digital converter at the signal output side after the operational amplifiers with which the amplified analog signals at the output of the operational amplifiers can be converted into a digital signal. The described aspect variant then has a computer, in particular a personal computer PC to which the digital signals can be transmitted for evaluation. The power spectrum calculation unit and the differentiation unit of the evaluation unit can be components of the computer in the variant described.

The exact embodiment of the force evaluating device in accordance with the invention and of the force evaluating method in accordance with the invention as well as further alternative embodiment possibilities will be described in the following by means of an example.

The apparatus in accordance with the invention and the method in accordance with the invention have a series of substantial advantages:
The quantitative detection and evaluation of the balance behavior of humans when standing is possible with the apparatus in accordance with the invention. A fall probability can ultimately be determined using the characteristics detected.
In particular the time development and the precision of the sensomotoric regulation of humans in the tandem stand can be detected as well as the capability of up to which time the tandem stand can be maintained.
The invention allows disorders in the spinal motoric system and in the supraspinal motoric system of humans to be modeled and to be quantified. The sources of the disorders can be isolated using the parameters detected by a variation of the test conditions (for instance, in addition to the tandem stand, examinations of a one-legged stand are also possible and the examinations can take place with the eyes open or closed). Changes can thus ultimately be detected by progressive examinations. Improvements in deficient positions of the system, e.g. of the motoric muscular performance, can then be monitored by means of training or treatment measures. The direct effect of disorders in the systems consists of a change in the fall probability which represents a substantial component of the risk of fracture.
The significance of the invention thus in particular lies in the possibility of objectively modeling and/or quantifying data, which are associated with the risk of falling and/or with the risk of fracture and which can otherwise only be raised anamnestically, as a measure of the voluntary and involuntary reaction capability. No external intervention is required for this on healthy people or sick people. The determination of age-specific and gender-specific standard values which are used as the basis for the assessment of health impairments takes place under standardized conditions.
The invention can in particular be used advantageously in the field of health care and in the examination of the physical fitness of the persons examined. The influence of drugs and alcohol can in particular also be determined objectively with the invention. Both unwanted side-effects and the influence of multimedication on persons can be examined in connection with the taking of medication. The area of use of the invention moreover also includes the diagnosis and monitoring of the driving capability of persons.
In the same way, the apparatus in accordance with the invention can be used as a diagnostic means in patients under rehabilitation measures, e.g. after apoplectic insult, and in persons who are suffering from diseases of the organ of balance.

### BRIEF DESCRIPTION OF DRAWINGS

In the following Figures belonging to the example, the same apparatus components are provided with identical reference numerals.

FIGS. 1A and 1B show the force receiving unit or the measuring unit of a force evaluating device in accordance with an exemplary embodiment of the present invention;

FIG. 2 shows a block diagram of the evaluation unit of the force evaluating device of FIGS. 1A and 1B;

FIG. 3 shows the output signals of the force inducers of the exemplary embodiment of FIGS. 1A and 1B detected over a time interval and their sum signal (total signal);

FIG. 4 shows the power spectrum which was calculated from the total signal shown in FIG. 3;

FIG. 5 shows the positive-value local maxima of the time derivation of the total signal of FIG. 3 sorted by size as well as the mean value calculated therefrom;

FIG. 6 sketches the movement of the dynamic center of gravity in the force inducer plane during the time interval of the measurement;

FIGS. 7A, 7B and 7C show three examples for the surface swept over by the dynamic center of gravity in the force inducer plane.

### DETAILED DESCRIPTION

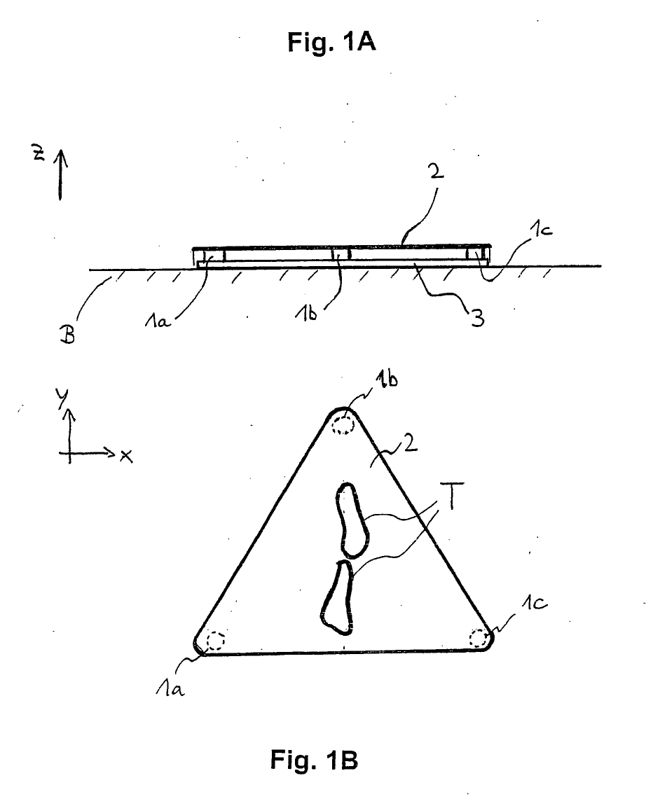

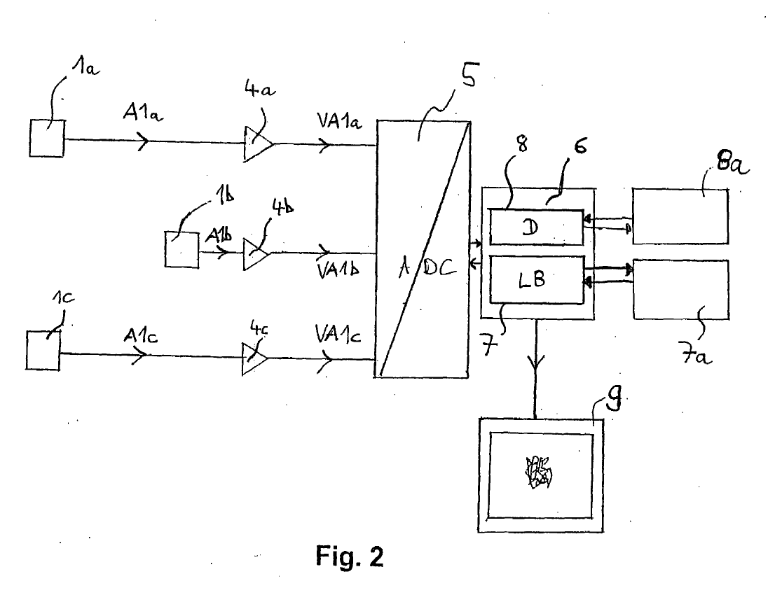

FIG. 1A shows a sectional view through a force evaluating device in accordance with the invention perpendicular to the force inducer plane. FIG. 1B shows a plan view of this force evaluating device or a plan view of the force inducer plane. In FIGS. 1A and 1B, only the components of the force evaluating device required for the detection of the force are shown (that is the components of the measuring unit of the force evaluating device). The evaluation unit for the evaluation of the detected forces is shown in the block diagram of FIG. 2 (see the following). The measuring unit and the evaluation unit communicate with one another to exchange data. The force evaluating device has a rigid, planar, triangular base plate 3. This is made of steel in the present case. The triangular form is defined by an equilateral triangle having an edge length here of 60 cm. The three triangle tips of the base plate are rounded. The base plate 3 has a thickness (perpendicular to the force inducer plane, that is in the z direction) of 1 cm. The base plate in the case shown is arranged above and adjoining a planar base surface B. Three force inducers 1a to 1c (in the following also alternatively designated as force sensors or load cells) are arranged on the base plate 3 in the region of the triangle tips. The load cells are screwed onto the base plate 3, but can, for example, also be adhered thereon. The three load cells are arranged in a plane (force inducer plane) in the form of an equilateral triangle. In this connection, each load cell is arranged slightly (a few cm) spaced apart from the peripheral rim of the base plate in the region of the triangle tip of the base plate so that the equilateral triangle formed by the three load cells 1a to 1c has a side length of approximately 55 cm. A support plate 2 likewise formed in the shape of an equilateral triangle is arranged above the three load cells 1a to 1c. The support plate is a break-proof plate on which a person weighing up to approx. 200 kg can stand and which is placed on the three load cells 1a to 1c in a non-slip manner. Forces or pressures acting on the support plate 2 from above (i.e. from the side opposite the load cells) can thus be transmitted proportionally to the three decoupled load cells 1a to 1c. The rigid support plate 2 which can be stood on has the same shape and size as the base plate 3 parallel to the force inducer plane (formed by the arrangement of the three force inducers 1). The support plate 2 is here a double ESG (safety glass) glass plate in accordance with DIN 1249 with a thickness of 16 mm.

The inducer axes of the three force inducers 1a to 1c are here arranged parallel to one another and in the z direction or perpendicular to the force inducer plane (x-y plane). The inducer axis of a force inducer is defined in that a force or force component acting on the force inducer 1 along this axis is determined or detected by the force inducer.

The force inducers 1a to 1c arranged between the base plate 3 and the support plate 2 are commercially available load cells. In the present case, cells are used which have a maximum load of 2000 N or 2 kN at a deformation or a nominal measurement path in the inducer axis direction of 0.2 mm. The load cells used have an accuracy class of <0.1% with respect to the forces detected. The nominal characteristic (sensitivity) or the signal voltage of the three cells amounts to 2 mV/V in each case. In the present case, the load cells “K-450” of the company “ATP Messtechnik+Waagen” are used.

Furthermore, the tandem stand T is sketched in FIG. 1B which a person standing on the support plate 2 should adopt during the detection and evaluation of the forces exerted on the force inducers 1a to 1c.

FIG. 2 shows a block diagram of the force evaluating device in accordance with the invention of FIGS. 1A and 1B in which both the cells of the measuring unit shown in FIGS. 1A and 1B and the components 4, 5, 6, 7, 8 of the evaluation unit connected downstream for the signal evolution can be seen. A respective operational amplifier 4a to 4c is connected at the signal output side downstream of each of the load cells 1a to 1c. If thus a person steps onto the support plate 2, the analog output signals A1a to A1c generated by the load cells 1a to 1c are transmitted to the operational amplifiers 4 by means of suitable signal lines. The operational amplifiers amplify the analog signals by a factor of 150 or 300 (different amplifier factors can also be used, however). The three now amplified analog signals VA1a to VA1c are supplied via suitable signal lines to an analog-digital converter ADC 5 connected downstream of the operational amplifiers 4 and having three input channels.

The ADC 5 is connected to a control computer (PC) 6 at the output side. In the present case, the ADC is of the type “miniLAB 1008” of the company Measurement Computing Corporation. In the present case, the operational amplifiers 4 and the ADC 5 are arranged between the base plate 3 and the support plate 2 within the triangle (not shown in FIGS. 1A and 1B) formed by the three load cells 1a to 1c. This has the advantage that the data lines for the transmission of the signals A1a to A1c or VA1a to VA1c are short. The ADC output signals are then transmitted over a USB line via a USB connection of the PC 6 into said PC. The ADC 5 can, however, also be made as a multi-channel data acquisition card which is arranged inside the computer 6. The ADC 5 thus generates USB-conforming signals which are fed into the control computer 6 from the electrical voltages which are generated on the basis of the pressure strain of the support plate 2 by the load cells 1a to 1c. The measured values of the load cells 1 or the corresponding voltages are digitized by the ADC 5 in real time.

However, this is not only a data transfer from the ADC 5 to the computer 6; the control computer 6 can vice versa also control the time window of the opening of the measurement channels of the ADC 5 via associated auxiliary software. The measuring interval via which the force values detected by means of the force sensors 1 are digitized and evaluated are thus fixed. The ADC 5 thus generates measured value time series of the three cells 1a to 1c which are available for further processing in the computer 6. The voltage supply of the ADC here takes place using a power pack that emits a stable 12 V.

In the present case, the power spectrum calculation unit 7 and the differentiation unit 8 are components of the computer 6. Both units 7, 8 each have a computing unit and a memory together with a command sequence 7a, 8a stored thereon. The memory and the computing unit of the power spectrum calculation unit 7 (also designated by LB in the Figure) and the differentiation unit 8 (also designated by D in the Figure) are identical here. Alternatively, separate memories and/or computing units can also be used, however, for both units.

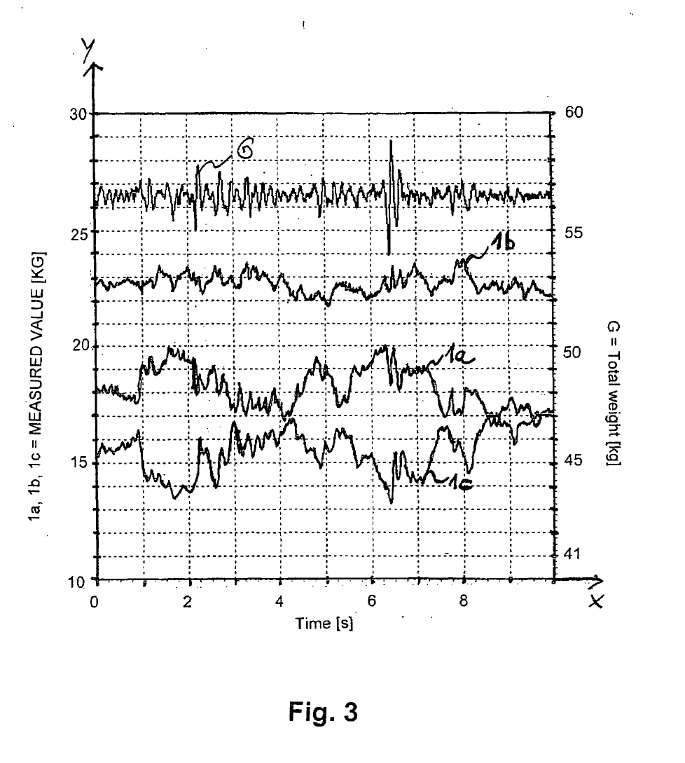

The three time measured value series over the measuring interval (that is the measurement curves over an adjustable time period or an adjustable time interval) detected and digitized by means of the three load cells 1, three operational amplifiers 4 and the ADC 5 are added in the computer 6 (see FIG. 3). The power spectrum calculation unit 7 calculates the power spectrum of the total signal from this added sum signal or total signal. This takes place using the command sequences 7a stored in the memory of the power spectrum calculation unit 7. In the same way, the differentiation unit 8 calculates the time derivation of the total signal in the measurement interval from the total signal. As described in the following, further characteristics are then calculated from this time derivation. The calculation of the differentiation unit 8 takes place with the command sequence 8a stored in the memory of the differentiation unit 8. The two described command sequences are here generated or compiled by means of the Delphi programming language. However, other programming languages such as C++ can also be used. The operating system used in the present case is Windows XP.

The results of the calculation can be represented graphically by the computer 6 on the output unit 9 (for example a monitor). The programs or command sequences described can moreover also check the calibrated nominal values of the sensors 1. The amplification factors of the pre-amplifiers, the scanning or sampling rates of the ADC 5 and the measuring duration or the time interval can moreover also be adapted to the data detection using the programs described. In the present case, the sampling rate amounts to 100 Hz (generally: at least 50 Hz); the amplification factors, as already described, to 150 or 300; and the measuring duration to 10 s. However, other values can also be used.

As FIG. 3 shows, the computer 6 calculates the sum of the measured values over the time interval from the individual sampled or measured force measurement values of the three force inducers 1a to 1c at each sampling time in the measuring interval. The total signal G hereby results. As the first characteristics, the total signal averaged over the time interval (here 10 s, x axis in FIG. 3) is determined. This average value G is a measure for the mass of the body or of the person on the support plate 2.

The total measurement value time series G shown in FIG. 3 or also the individual measurement value time series 1a to 1c or any desired sum combinations of these three measurement value time series can now be selected for further processing in the power spectrum calculation unit 7 or the differentiation unit 8.

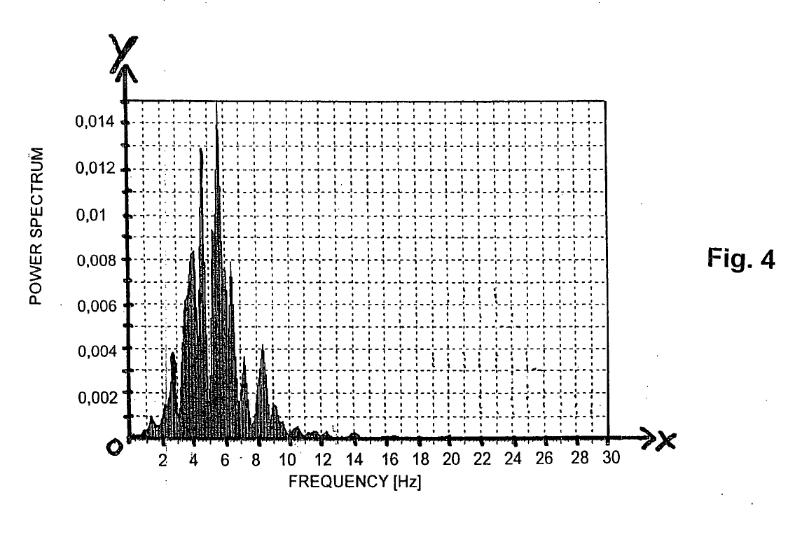

FIG. 4 thus shows the power spectrum of the total signal G of FIG. 3. This power spectrum was calculated by means of the power spectrum calculation unit together with the command sequence 7a stored thereon. The basis for the calculation here is a sampling of the analog output signals VA1a to VA1c by the ADC 5 using a sampling rate of at least 50 Hz. In the present case, sampling was carried out at 100 Hz. The minimum value of 50 Hz corresponds approximately to four times the frequencies to be expected. At such a sampling rate, low force pulses arise which are predominantly generated by the flexor group of the foot musculature and the peroneus muscles of the person standing on the support plate 2 during the measurement interval.

The time series G of the force sum of the three sensors 1a to 1c was subjected to a frequency partition for the calculation of the power spectrum. This was carried out in the present case by means of the method of Fourier transformation, here in particular of fast Fourier transformation FFT. Alternatively to the FFT method, however, a frequency partition can also be carried out using the method of maximum entropy by means of the power spectrum calculation unit 7 (with respect to the method of maximum entropy and to the fast Fourier transformation, see for example “Numerical Recipes” in Pascal: The art of Scientific Computing, William H. Preis, Brian P. Flannery, Saul A. Teukolsky, William T. Vetterling, Cambridge University Press, 1989).

The method of maximum entropy offers the advantage of a higher resolution in this connection.

The power spectrum calculation unit 7 thus performs an analysis of the frequency portions in the force pulses (spectral analysis) by means of the program code 7a. The power spectrum determined is shown in FIG. 4 in the range from 0 Hz to 30 Hz. As the Figure shows, muscular force actions are mainly found in the region of 0 to approximately 13 Hz.

The power spectrum can then be further evaluated by means of the unit 7. Arithmetical or geometrical mean values of the frequencies or average frequencies which occur can thus e.g. be determined. They permit a statement on the reaction capability of the test person: The higher the average frequencies, the higher the reaction capability of the person.

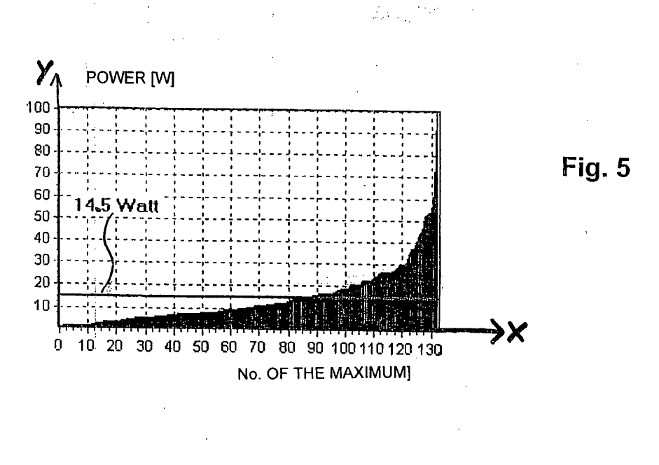

FIG. 5 shows an example for the characteristics determined by means of the differentiation unit 8 and the program sequence 8a. If, as in the present case, the force pulses of the sum curve or of the total signal G (cf. FIG. 3) have a sufficiently high resolution due to the sampling or scanning rate used (100 Hz here, generally sampling rates between 50 and 250 Hz are preferably to be used), the total signal G shown in FIG. 3 can be differentiated numerically by time. The numerical differentiation can be carried out, as described briefly in the following, by means of a simplified support point method. A number of support points is first defined for this purpose. It preferably amounts to between 5 and 15 support points. 10 support points were used in the present case. Starting from each measured value or sampled value of the sum curve G shown in FIG. 3, the local pitch between this measured value and the measured value following this measured value in time by the said number of support points is determined. If the pitch determined from the two measured values spaced apart by the named number of support points is positive, the two measured values by means of which the pitch is determined are offset backward in each case by one measured value in time in the total sum curve G (that is the respective measured values being later by one sampled value are used for the pitch determination) and the local pitch is again determined. If this local pitch is larger than that determined in the preceding step, the process is continued, i.e. the measured value pair by means of which the pitch is determined is displaced backward in time by a further measured value. The process stops when the locally determined pitch no longer increases, decreases again or becomes negative. The local pitch previously determined, that is in the penultimate step (that is the local maximum value), is flagged as the peak pulse value in the memory. The process now proceeds for so long until the local pitch increases again and assumes a positive value. Subsequently, as described, the locally present peak pulse value is again determined and flagged. A set of peak pulse values thus results on the described sampling of the total sum curve G, said peak pulse values in each case representing locally occurring maximum pitches having a different positive value. This set can subsequently be analyzed. It is thus possible, for example, to prepare a histogram of the set and to calculate a mean value or a mean peak pulse power. Alternatively, as shown in FIG. 5, the individual recorded peak pulse values can also be sorted by size (x axis in FIG. 5). In the region of the origin, the smallest (positive) local peak pulse values are then entered, whereas the local peak pulse values which become larger are entered on the x axis as the distance from the origin increases. Here, too, the mean peak pulse value can then be determined (horizontal line).

The described peak pulse values are now a measure for the peak power which occurs at the corresponding point in time and which is exerted by the person standing on the support plate 2 to maintain balance. This results from the following consideration: the differentiation of a force by time results in the muscular performance when the work performed is known (work=force*distance). The performance is physically defined as the work performed per unit of time. However, in the present case, the work results from the force against the “spring travel” of the force inducer 1. The differentiation unit 8 or the program 8a can thus filter out the local maximum peak pulse powers of the individual positive force increases in the manner described. The mean peak pulse power value can then be calculated from these pulse peak power values whose spectrum can be shown in a sorted manner, as shown in FIG. 5.

The required power which is required to return a body to the neutral center of gravity or to the balanced position naturally also depends on the body mass. The ratio of the named mean peak pulse power value and the time average of the sum curve G over the measurement interval is thus calculated as a further characteristic. The named ratio or the division of the mean peak pulse power value by the body mass thus results in the mean peak pulse power per body mass (specific mean peak pulse power). This value is then comparable independently on the body mass of the respectively examined individual.

The mean peak pulse power (horizontal line) or the specific mean peak pulse power (mean peak pulse power per body mass) calculated from this is a measure for the force effort and/or the performance effort a person standing on the support plate 2 has to expend to maintain balance.

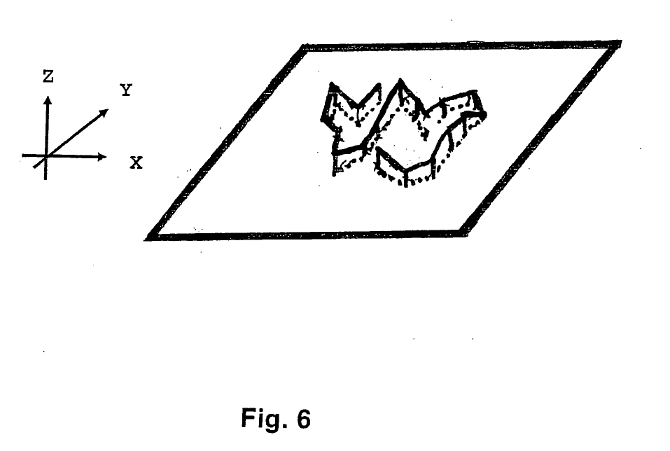

The calculation of a further characteristic is shown in FIG. 6. This Figure sketches how a calculation of the position of the dynamic center of mass of the person standing on the support plate 2 is determined by means of the signal differences of the three decoupled load cells 1a to 1c. The dynamic center of gravity can be composed of a static portion (static center of gravity of the person) resulting from the person's mass and of a portion resulting from a force exerted on the support plate 2 by the person by means of muscular forces. For this purpose, an orthogonal coordinate system is first selected whose x-y plane corresponds to the force inducer plane K. The x-y plane of the coordinate system thus lies in the plane which is formed by the three contact points of the load cells 1a to 1c. The spatial change of the dynamic center of gravity or the track of the center of gravity of the force vector is now calculated via the measurement interval or the detected time interval from the body weight and the interaction of the muscular forces (the latter represents the dynamic portion of the center of gravity). In this connection, the calculation takes place for the projection of the dynamic center of gravity onto the x-y plane or the force inducer plane. The calculation can take place for each sampling time using the simple formula

$${\sum }_{i = 1}^{N} \frac{{m}_{i}}{{m}_{ges .}} {{r}^{->}}_{i}$$

for example. Here, N is the number of force inducers (here N=3); is the ith of these force inducers (i=1, 2 or 3); mᵢ is the measured value of the ith force inducer at the time of measurement; m_{ges}. is the sum over the measured values of the three force inducers at this time; and \vec{r}ᵢ is the spatial vector of the ith force inducer in the orthogonal coordinate system shown. If the person were to stand absolutely still on the support plate 2, the projection of the dynamic center of gravity or of the force vector onto the force inducer plane K would result in exactly one point in the force inducer plane (no dynamic portion, only static center of gravity portion). A person standing on the support plate 2 can, however, not stand completely still so that fluctuations in the center of gravity of the person result in muscular correction forces. The dynamic center of gravity or its projection onto the x-y plane thus performs a migration during the measured interval. This is shown in FIG. 6 (dotted line: track of the dynamic center of gravity). In addition, a further characteristic in the third dimension (i.e. on the z axis) is sketched here. This value is determined from the sum of the signals of the three cells generated at the respective sampling time (that is from a value of the sum curve G in FIG. 3) less the time average of the sum curve G over the measured interval (z: amount of the force vector perpendicular to the plate).

The time development of the spatial change of the dynamic center of gravity in the platform plane or the force transducer plane K is thus calculated which results from the migration of the center of mass and the time-variable acceleration forces of the musculature for the maintenance and correction of balance. This spatial change (x(t), y(t)) (t=time) is thus determined for all sampled times or measurement times in the measurement interval and is stored by the computer 6. The regulative acceleration force which results from subtraction of the static body weight from the instantaneous total pulses registered by the load cells or from the sum of the single pulses is calculated in real time and stored as the third component (z(t) component). The static body weight is calculated only after the end of the measurement (in the same manner as the sum curve G from the three signals or the three individual measured value time series) when the allocated buffer memory in the PC is read out.

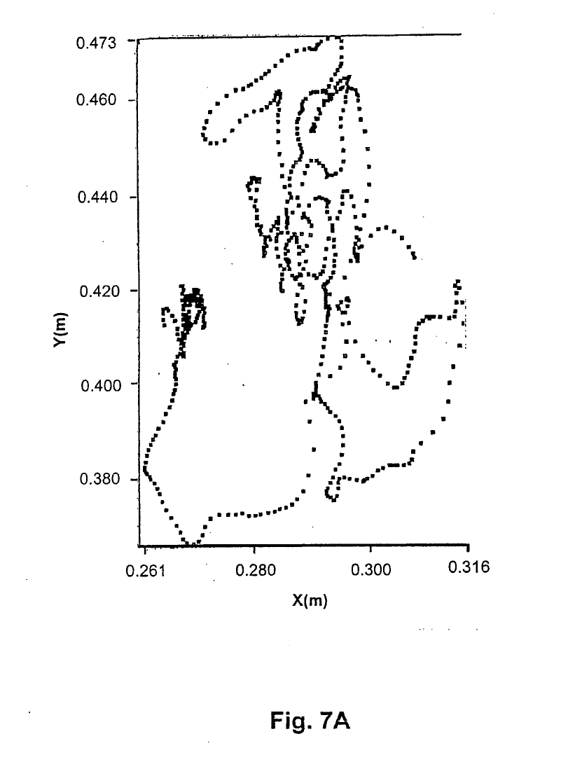

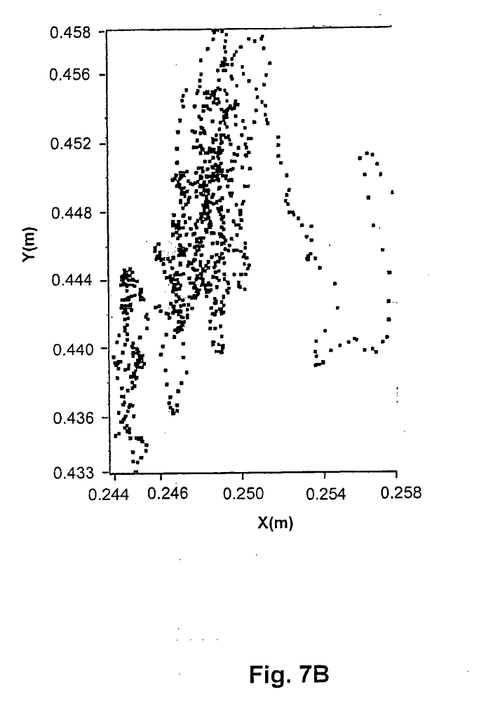

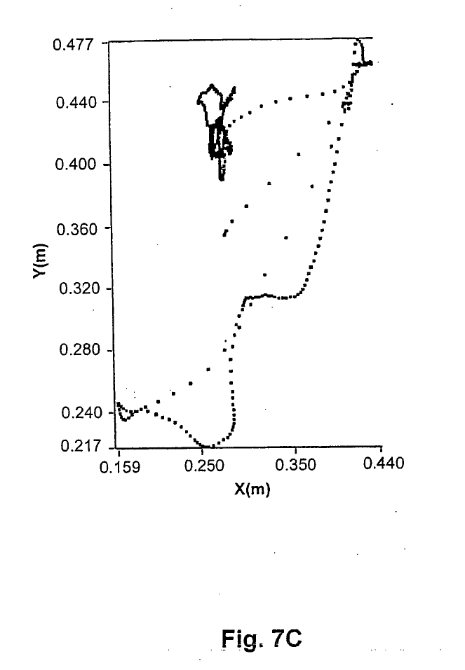

FIGS. 7A, 7B and 7C show three examples for the surface swept over by the migration of the center of gravity position (x, y) in the plane K during the measurement interval. The surface swept over is quantitatively detected by means of the evaluation unit, here by means of the PC. In the case shown in FIG. 7A, the surface amounts to approximately 5.5*10.7 cm². In the case shown in FIG. 7B, the surface amounts to only 1.4*2.5 cm². In FIG. 7c, the surface swept over amounts to approximately 28.1*26 cm². The correspondingly swept over surface is now a measure for the capability of the respective person (A or B or C) to maintain balance on the support plate 2 during the measurement interval. The smaller the area swept over, the better this capability. The areas swept over or the metric extents in the coordinate system in the x-y plane of different persons can now be compared with one another. This allows conclusions, for example, on possible diseases of the examined neuromuscular functions. The three results shown thus document clear differences between a healthy person without risk of a fall (B), a person with a low risk (A) and a person at great risk of a fall (C). It is to be expected here that the area swept over or the metric extent in the x-y diagram is, in a first approximation, independent of the age of the examined person, but very dependent on other influences such as also the taking of medication, taking of alcohol and/or drugs, disorders in maintaining balance in general and also after apoplective insult or diseases of the organ of balance.

In summary, there are thus calculated with the present apparatus or with the present method:
the track of the dynamic center of gravity within the step plane or force inducer plane;
the power spectrum or the frequency spectrum of the muscular activities;
the muscular performance from the time development G of the forces or its derivation; and
a measure for the specific muscular performance (specific mean peak pulse performance).

This is done in the provoked unstable stance, preferably in the tandem stand or standing on one leg. The optical perception of the person can be disabled here (closing the eyes), which makes maintaining balance more difficult.

1. A force evaluating device for determining and evaluating forces acting on force inducers in a time-resolved manner, comprising:
at least three force inducers arranged in a plane (force inducer plane), each force inducer having a signal input and a signal output;
a rigid walkable support plate coupled to the force inducers, the force inducers being arranged at a side of and adjacent to the support plate, the forces from a compressive force acting on the support plate resulting at the force inducers being detected in the time-resolved manner; and
an evaluation unit connected to the force inducers at the signal output side, the evaluation unit including a power spectrum calculation unit, the power spectrum calculation unit at least one of (i) calculating a power spectrum of a total signal formed from a sum of the output signals of at least one of the force inducers and (ii) having a differentiation unit calculating a time derivation of the total signal and further parameters derived therefrom.

2. The force evaluating device of claim 1, wherein the power spectrum calculation unit has a first calculation unit and a first memory with a first command sequence stored thereon, the first calculation unit calculating the power spectrum.

3. The force evaluating device of claim 2, wherein the differentiation unit has a second calculation unit and a second memory with a second command sequence stored thereon, the second calculation unit calculating the time derivation and the further characteristics.

4. The force evaluating device of claim 3, wherein the first and second calculation units are identical.

5. The force evaluating device of claim 3, wherein the first and second memories are identical.

6. The force evaluating device of claim 1, wherein inducer axes of the force inducers are arranged substantially parallel to one another and substantially perpendicular to the force inducer plane, each inducer axis being defined so that one of a force and a force component acting on the force inducer along this axis is determined by the force inducer.

7. The force evaluating device of claim 1, wherein exactly three force inducers are arranged in the force inducer plane in a form of a triangle.

8. The force evaluating device of claim 1, wherein exactly three force inducers are arranged in the force inducer plane in a form of an isosceles triangle.

9. The force evaluating device of claim 1, wherein exactly three force inducers are arranged in the force inducer plane in a form of an equilateral triangle.

10. The force evaluating device of claim 1, wherein the support plate has a triangular shape.

11. The force evaluating device of claim 10, wherein the triangular shape is an equilateral triangle.

12. The force evaluating device of claim 10, wherein a side length of the triangle is at least one of (i) greater than 50 cm and (ii) less than 80 cm.

13. The force evaluating device of claim 10, wherein a side length of the triangle is at least one of (i) greater than 55 cm and (ii) less than 75 cm.

14. The force evaluating device of claim 10, wherein the support plate has a pressure force capacity of at least one of (i) greater than 0.5 kN and (ii) less than 5 kN.

15. The force evaluating device of claim 10, wherein the support plate has a pressure force capacity of at least one of (i) greater than 1 kN and (ii) less than 2 kN.

16. The force evaluating device of claim 1, further comprising:
a base plate arranged on a side remote from the support plate and adjacent to the force inducers.

17. The force evaluating device of claim 16, wherein the base plate has at least one of a same shape, a same extent, and a same compressive force capacity as the support plate.

18. The force evaluating device of claim 1, wherein at least one of the force inducers is a piezo sensor.

19. The force evaluating device of claim 1, wherein at least one of the force inducers has at least one of (a) a maximum load value of at least one of (i) greater than 0.5 kN and (ii) less than 5 kN; (b) a measuring accuracy of less than 1%; and (c) one of a nominal characteristic and a sensitivity of at least one of (i) greater than 1 mV/V and (ii) less than 10 mV/V.

20. The force evaluating device of claim 19, wherein the maximum load value is at least one of (i) greater than 1 kN and (ii) less than 2 kN.

21. The force evaluating device of claim 19, wherein the measuring accuracy is less than 0.1%.

22. The force evaluating device of claim 19, wherein one of a nominal characteristic and a sensitivity is 2 mV/V.

23. The force evaluating device of claim 1, wherein at least one of the force inducers has a maximum deformation (nominal measurement path) along its inducer axis of less than 1 mm.

24. The force evaluating device of claim 1, wherein at least one of the force inducers has a maximum deformation (nominal measurement path) along its inducer axis of less than 0.3 mm.

25. The force evaluating device of claim 1, wherein the evaluation unit has at least one operational amplifier connected to at least one of the force inducers at a signal output side.

26. The force evaluating device of claim 25, wherein the at least one operational amplifier is connected to each of the force inducers at the signal output side.

27. The force evaluating device of claim 1, wherein the evaluation unit has an analog/digital converter ADC, the force inducers being connected to the ADC at the signal output side.

28. The force evaluating device of claim 27, wherein the ADC is a data acquisition card.

29. The force evaluating device of claim 28, wherein the data acquisition card has a number of channels corresponding to the number of the force inducers.

30. The force evaluating device of claim 1, wherein the evaluation unit has a computer.

31. The force evaluating device of claim 30, wherein the computer is a personal computer PC.

32. The force evaluating device of claim 1, wherein the evaluation unit has an analog/digital converter ADC and a computer, the computer being connected to the signal output side of the ADC.

33. The force evaluating device of claim 1, wherein the force evaluating device is used to one of determine and monitor at least one of driving capabilities of persons, an influence of at least one of alcohol and drugs on motoric skills of persons, and characteristics for physical fitness of persons.

34. A force evaluating method with which forces acting on force inducers is determined and evaluated in a time-resolved manner, comprising:
arranging at least three force inducers comprising a signal input and a signal output in a plane (force inducer plane) at a side of and adjacent to a rigid, walkable support plate and coupled to the support plate such that the forces from a compressive force acting on the support plate resulting at the force inducers are detected by the force inducers in a time-resolved manner;
exposing the support plate to a pressure on a remote side from the force inducers over a predetermined time interval;
forming a time-resolved total signal for the time interval from a sum of the output signals of at least one of the force inducers resulting from the pressure exertion at the force inducers; and
calculating at least one of (i) the power spectrum of the total signal and (ii) the time derivation of the total signal and further characteristics derived therefrom.

35. The force evaluating method of claim 34, wherein at least one of the total signal, the power spectrum, the time derivation, and the further derived characteristics are calculated using a computer-assisted evaluation unit connected to the force inducers at the signal output side.

36. The force evaluating method of claim 34, wherein the force evaluating unit is connected to the force inducers at the signal output side, the evaluation unit having a power spectrum calculation unit, the power spectrum calculation unit at least one of (i) calculating the power spectrum of a total signal formed from a sum of the output signals of at least one of the force inducers and (ii) having a differentiation unit calculating the time derivation of the total signal and further parameters derived therefrom.

37. The force evaluating method of claim 34, wherein the pressure load is generated by a person at least one of walking on the support plate and standing on the support plate.

38. The force evaluating method of claim 34, wherein the force inducers detect one of forces and force components acting on the force inducers substantially perpendicular to the force inducer plane.

39. The force evaluating method of claim 34, wherein exactly three force inducers are arranged in the force inducer plane in a form of a triangle.

40. The force evaluating method of claim 34, wherein exactly three force inducers are arranged in the force inducer plane in a form of an isosceles triangle.

41. The force evaluating method of claim 34, wherein exactly three force inducers are arranged in the force inducer plane in a form of an equilateral triangle.

42. The force evaluating method of claim 34, wherein one of (i) the sum of the output signals and (ii) the measured value time series of all force inducers form one of the total signal and the total value time series forming the total signal.

43. The force evaluating method of claim 34, further comprising:
forming a time average of the total signal over the time interval for the determination of the mean pressure load on the support plate.

44. The force evaluating method of claim 34, wherein the output signal of at least one force inducer is sampled at a sampling rate of more than 40 Hz.

45. The force evaluating method of claim 34, wherein the output signal of at least one force inducer is sampled at a sampling rate of at least one of greater than 50 Hz and less than 250 Hz.

46. The force evaluating method of claim 37, wherein an instantaneous position of a dynamic center of gravity of the person projected onto the force inducer plane is determined from the output signals of the force inducers over the time interval, the dynamic center of gravity being composed of a static portion resulting from a mass of the person (static center of gravity of the person) and a portion resulting from the force exerted on the support plate from muscular forces by the person.

47. The force evaluating method of claim 46, wherein the instantaneous position of the dynamic centre of gravity is calculated in accordance with
$${\sum }_{i = 1}^{N} \frac{{m}_{i}}{{m}_{ges .}} {{r}^{->}}_{i} ,$$
where N is a number of the force inducers and i is an ith force inducer and where, at the time observed, mᵢ is a measured value of the ith force inducer, m_{ges} is the sum over measured values of the N force inducers, and \vec{r}ᵢ is a spatial vector of the ith force inducer in an orthogonal coordinate system.

48. The force evaluating method of claim 46, wherein a surface swept over during the time interval is determined from one of the instantaneous position of the dynamic center of gravity and a movement track in the force inducer plane.

49. The force evaluating method of claim 48, further comprising:
calculating a ratio of a first person swept-over surface and a second person swept-over surface different from the first person swept-over surface.

50. The force evaluating method of claim 34, wherein the power spectrum of the total signal is calculated using at least one of a Fourier transformation, a fast Fourier transformation and a method of maximum entropy.

51. The force evaluating method of claim 34, wherein the calculated power spectrum is evaluated with respect to frequencies contained therein.

52. The force evaluating method of claim 34, wherein at least one of an arithmetical mean of frequencies, a geometrical mean of frequencies and a maxima contained in the power spectrum is calculated.

53. The force evaluating method of claim 52, wherein a positive value local maxima of the time derivation of the total signal are determined over the time interval.

54. The force evaluating method of claim 52, wherein a histogram is prepared from the maxima in accordance with respective functional values.

55. The force evaluating method of claim 52, wherein the maxima are sorted according to a size of the respective functional values.

56. The force evaluating method of claim 52, wherein a mean value of the functional values of the maxima is determined.

57. The force evaluating method of claim 54, wherein each maximum is multiplied by a weighting factor prior to the preparation of the histogram the weighting factor of a maximum being calculated in each case from characteristics of the force inducers.

58. The force evaluating method of claim 55, wherein each maximum is multiplied by a weighting factor prior to the size sorting, the weighting factor of a maximum being calculated in each case from characteristics of the force inducers.

59. The force evaluating method of claim 57, wherein the weighting factor of the maximum is calculated in each case from the nominal characteristics of the force inducers and the output signals emitted by the force inducers at the time corresponding to the local maximum.

60. The force evaluating method of claim 58, wherein the weighting factor of the maximum is calculated in each case from the nominal characteristics of the force inducers and the output signals emitted by the force inducers at the time corresponding to the local maximum.

61. The force evaluating method of claim 43, wherein the time average of the total signal is formed over the time interval for the determination of the mean pressure load on the support plate, a ratio of the mean value of the functional values of maxima and the time average of the total signal being calculated as the characteristic.

62. The force evaluating method of claim 61, wherein the pressure load is generated by a person at least one of walking on the support plate and standing on the support plate, a ratio being calculated from the mean value of the functional values of the maxima determined for a first person and from the mean value of the functional values of the maxima determined for a second person different from the first person.

63. The force evaluating method of claim 48, wherein one of a presence and an absence of at least one of
(a) a disorder in at least one of a spinal motoric system and a supraspinal motoric system,
(b) one of a balance disorder and a disorder in maintaining balance in standing,
(c) a gait disorder,
(d) a reduction in at least one of the strength and a muscular power, and
(e) a cognitive reduction with respect to the environment,
(f) a reduction in visual acuity,
(g) a side-effect of taking medication, and
(h) an influence of multimedication
is diagnosed in the person using the surface swept over.

64. The force evaluating method of claim 63, wherein the reduction corresponds to at least one of the strength and the muscular power in lower extremities.

65. The force evaluating method of claim 63, wherein a quantitative measure is determined using the surface swept over for the disorder in at least one of the spinal motoric system and the supraspinal motoric system, one of the balance disorder and the disorder in maintaining balance when standing, the gait disorder, the reduction in at least one of the strength and the muscular power, the cognitive reduction with respect to the environment, the reduction in visual acuity, the side-effect when taking medication, and the influence of multimedication.

66. The force evaluating method of claim 63, wherein a quantitative measure is determined using the surface swept over.

67. The force evaluating method of claim 48, wherein a qualitative measure is determined using the surface swept over for the person for at least one of the regulation of the motoric and sensory system, for at least one of the sensomotoric regulation of the center of gravity and for at least one of the balance regulation and for the voluntary and involuntary reaction of the person, and for at least one of a probability of falling of the person and for the physiological effects of the taking of medication by the person.

68. The force evaluating method of claim 63, wherein one of a presence and an absence of a disorder in at least one of the spinal motoric and supraspinal motoric system, one of a balance disorder and a disorder in maintaining balance in standing, of a gait disorder, at least one of a reduction in the strength and muscular performance, of a cognitive reduction with respect to the environment, a reduction in visual acuity, at least one of a side-effect of taking medication and an influence of multimedication is diagnosed in the person by means of at least one of the calculated power spectrum, the calculated time derivation, and the derived characteristics.

69. The force evaluating method of claim 68, wherein a quantitative measure is determined using at least one of the calculated power spectrum, the calculated time derivation and the derived characteristics for the disorder in at least one of the spinal motoric and supraspinal motoric system, the balance disorder or the disorder in maintaining balance when standing, the gait disorder, at least one of the reduction in the strength and muscular power, the cognitive reduction with respect to the environment, the reduction in visual acuity, the side-effect of at least one of taking medication and the influence of multimedication.

70. The force evaluating method of claim 68, wherein a quantitative measure is determined using the surface swept over.

71. The force evaluating method of claim 34, wherein a quantitative measure is determined on the basis of the calculated power spectrum, of at least one of the calculated time derivation and of the derived characteristics for the person for at least one of the regulation of the motoric and sensory system, for the sensomotoric regulation of at least one of the center of gravity, the balance regulation, and the voluntary and involuntary reaction of the person, for at least one of the probability of falling of the person and the physiological effects of the taking of medication by the person.

72. Use of a force evaluating method according to claim 34.
